(19) United States
(12) Patent Application Publication
(10) Pub. No.: US 2018/0015241 A1
(43) Pub. Date: Jan. 18, 2018

(54) SPACER CHAMBER

(71) Applicant: Diego Jaroslavsky, Ciudad Autonoma de Buenos Aires (AR)

(72) Inventor: Diego Jaroslavsky, Ciudad Autonoma de Buenos Aires (AR)

(21) Appl. No.: 15/637,057

(22) Filed: Jun. 29, 2017

Related U.S. Application Data
(63) Continuation-in-part of application No. 14/458,297, filed on Aug. 13, 2014, now abandoned.

(51) Int. Cl. A61M 15/00

(52) U.S. Cl. CPC A61M 15/0086; A61M 2205/02; A61M 2205/0216

(57) ABSTRACT

The present invention refers to a single piece spacer chamber, a base end and a tip end, the base end having a diameter larger than a diameter of the tip end, single piece chamber is made entirely of a flexible material. A medication container is connected to the tip end. The chamber has a truncated cone shape, a plurality of external zigzag popoid folds circumferentially formed through the external side of the chamber, and a plurality of internal folds circumferentially formed through the internal side of the chamber. The popoid folds have a size that decreases from the base end to the tip end. In a collapsed position each popoid folds against an adjacent popoid and fits entirely inside the adjacent popoid.

### CROSS-REFERENCE TO RELATED APPLICATIONS

This application is a continuation-in-part of patent application Ser. No. 14/458,297 filed Aug. 13, 2014, entitled SPACER CHAMBER, pending, the disclosure of which is incorporated herein by reference.

### FIELD OF THE INVENTION

The present invention refers to a spacer chamber that helps to disperse a medical drug to a patient. More specifically, the present invention relates to a spacer chamber that can collapse into a very little device when it is transported or stored.

### BACKGROUND INFORMATION

A series of spacer chambers having different shapes and mechanisms are commercially known to facilitate the pharmaceutical drug administration by respiratory inhalation. The majority of them being variations of the ones used in children or adults.

Many types of spacer chambers have been developed throughout the years. The known spacer chambers have different volumes and sizes. Unfortunately, a lot of the known chambers are difficult to transport in the case of being personally used.

Collapsible spacer chambers have been developed, with different types of structures, for example, including bellows (accordion shape) or with rigid rings. Unfortunately, they are difficult to properly extend, especially at the time the patient needs the medication.

In addition, the spacer chambers of the prior art are made of plastic, which is a material that generates a lot of static electricity and attracts the aerosol particles. To prevent this problem, the prior created spacer chamber is made of stainless steel. The prior art also added additives that do not charge with static electricity to prevent the adhesion of particles to the stainless steel chambers.

### SUMMARY OF THE INVENTION

The present invention refers to a spacer chamber for administering a therapeutic agent spray. The spacer chamber includes:

a one piece body having a base end, a tip end, and a popoid section,

wherein the one piece body is made of a flexible material and has a shape of a cone;

wherein a diameter of the base end is larger than a diameter of the tip end;

wherein the popoid section includes a plurality of popoids, each popoid includes a first set of frustoconical portions facing opposite to a second set of frustoconical portions to form corrugations in the body;

wherein the first and second set of frustoconical portions of the popoid near to the base end have a greater diameter than the diameter of the first and second set of frustoconical portions of the popoid near to the tip end; and

wherein when the tip end moves toward the base end, the frustoconical portions maintain their shape, while the frustoconical portions become inverted to fit within the associated frustoconical portions, thus, each popoid collapses upon itself and each popoid beginning from the tip end collapses completely inside an adjacent popoid shortening the length of the body.

### BRIEF DESCRIPTION OF THE DRAWINGS

FIG. 1 is a perspective view of the spacer chamber according to the prior art;

FIG. 2 is a side view of the spacer chamber according to an embodiment of the present invention;

FIG. 3 is a front view of the spacer chamber of FIG. 2;

FIG. 4 is a side view of the spacer chamber of FIG. 2 in an extended position;

FIG. 5 is a side view of the spacer chamber of FIG. 2 in a semi-folded position;

FIG. 6 is a side view of the spacer chamber according to an embodiment of the present invention including the chamber joined by their bases and also showing the chamber in an extended position;

FIG. 7 is a side view of the spacer chamber of FIG. 6 showing the chamber in a collapsed position;

FIG. 8 shows a side view of the spacer chamber according to one embodiment of the present invention showing a spacer chamber for outpatient treatment showing the chamber in an extended position;

FIG. 9 shows a top view of the spacer chamber of FIG. 8 showing the chamber in a collapsed position;

FIG. 10 shows a cross sectional view of the spacer chamber taken along line A-A of FIG. 9 showing the chamber in a collapsed position;

FIG. 11 shows a side view of the spacer chamber according to one embodiment of the present invention showing a spacer chamber in use with a mechanical ventilation;

FIG. 12 shows a left side view of the spacer chamber of FIG. 11;

FIG. 13 shows a top view of the spacer chamber of FIG. 11;

FIG. 14 shows a cross sectional view of the spacer chamber taken along line C-C of FIG. 13;

FIG. 15 shows a top view of the spacer chamber according to an embodiment of the present invention;

FIG. 16 is a cross sectional view of the spacer chamber of FIG. 15 taken along line A-A′ showing the extended chamber and the mask connected to the chamber;

FIG. 17 is a top view of the spacer chamber according to an embodiment of the present invention showing the extended chamber in use with a mechanical ventilation and including a threaded base end and a threaded tip end;

FIG. 18 shows a cross sectional view of the spacer chamber of FIG. 17 taken along line A-A′;

FIG. 19 is a side view of the spacer chamber according to an embodiment of the present invention showing the extended chamber spacer chamber including a flap to extend the chamber, a thread end, and orifices in the threaded end; and

FIG. 20 shows a top view of the spacer chamber of FIG. 20.

### DETAILED DESCRIPTION OF THE INVENTION

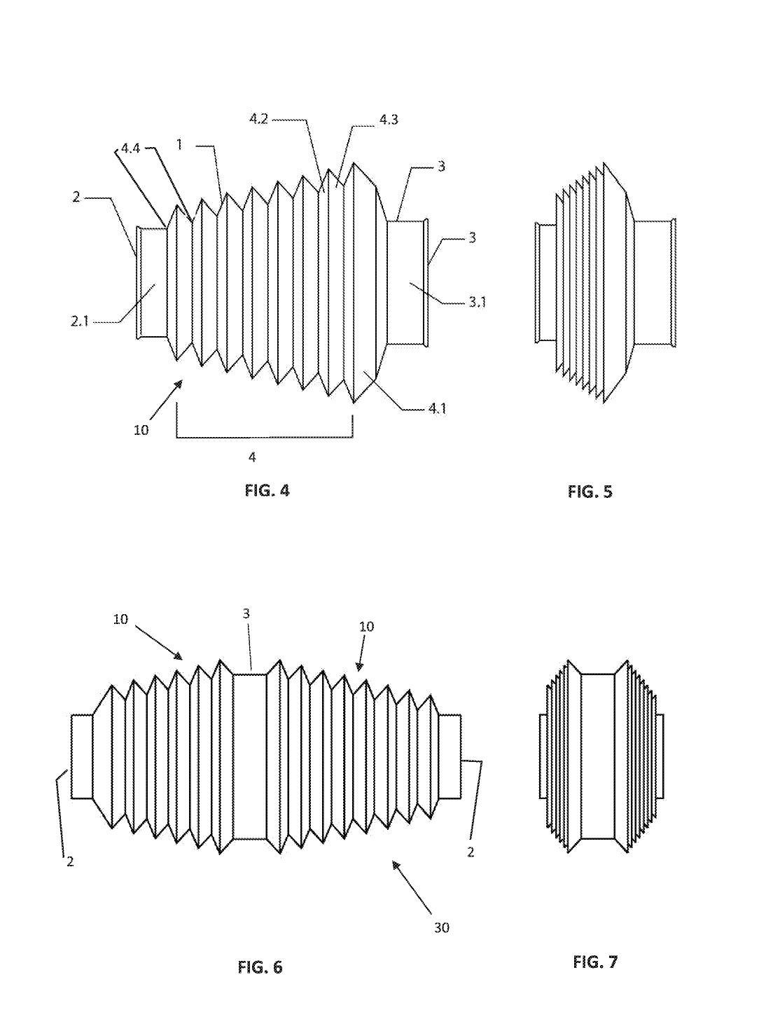

FIGS. 2 to 5 illustrate a spacer chamber 10 including a one piece body 1 having a base end 3, a tip end 2, and a popoid section 4. The one piece body 1 has a shape of a cone with the diameter 3.1 of the base end 3 larger than the diameter 2.1 of the tip end 2.

The body 1 is of one piece construction and is preferably formed from a suitable anti-static material, such as polypropylene.

The popoid section 4 includes a plurality of popoids 4.1. Each popoid 4.1 includes a first set of frustoconical portions 4.2 facing opposite to a second set of frustoconical portions 4.3 to form a corrugation 4.4 in the body 1.

The first and second set of frustoconical portions 4.2, 4.3 of the corrugation 4.4 near to the base end 3 have a greater diameter than the diameter of the first and second set of frustoconical portions 4.2, 4.3 of the popoid 4.1 near to the tip end 2.

When the tip end 2 is moved toward the base end 2, the frustoconical portions 4.3 maintain their shape, while the frustoconical portions 4.2 become inverted to fit within the associated frustoconical portions 4.3 Thus, each popoid 4.1 will collapse upon itself. In addition, each popoid 4.1 beginning from the tip end 2 to the base end 3, will collapse completely inside the adjacent popoid 4.1 shortening the length of the body 1.

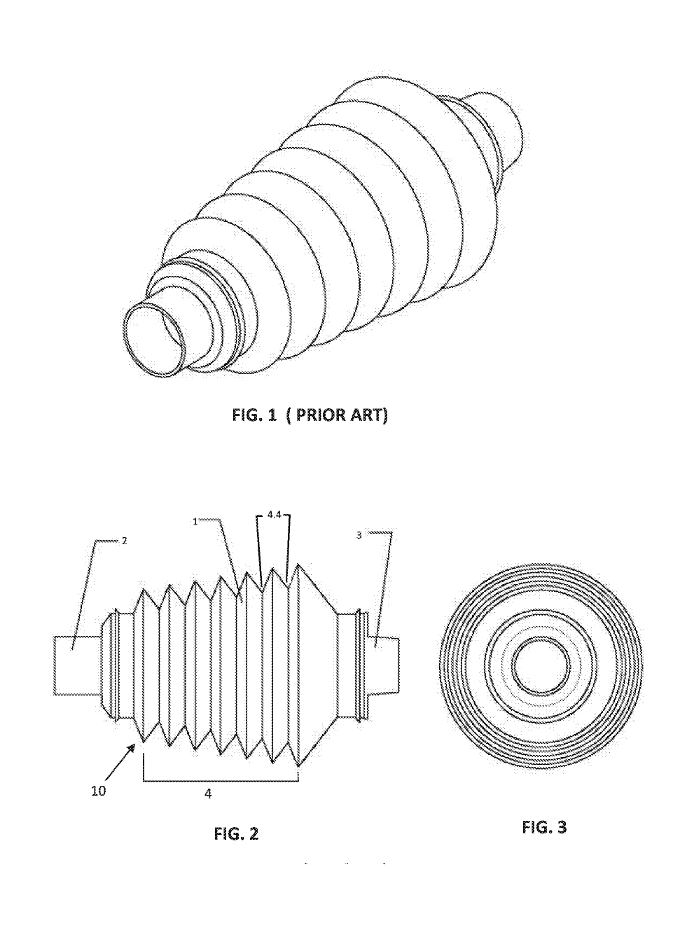

The collapsing of each one of the frustoconical portions 4.2 into the corresponding frustoconical portions 4., allows the popoid 4.1 to keep their mechanical strength longer since the material forming the popoid 4.1 is expanded to a lesser degree than the popoid of the prior art showed on FIG. 1.

FIGS. 6-7 shows a spacer chamber 30 according to an embodiment of the present invention including to chambers 10 joined by their bases.

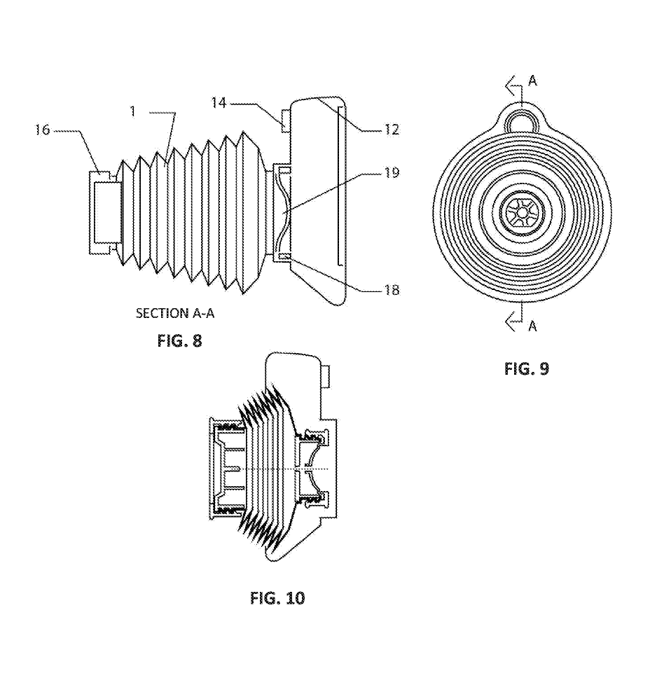

FIGS. 8-10 show the spacer chamber 10 according to the present invention showing a spacer chamber for use during outpatient treatment. A mask 12 for the patient is connected to the base end 3. The mask 12 includes a threaded end 18 to connect to the base end 3, an exhalation valve 14, and an aspiration valve 19. A threaded support 16 for aerosol is connected to the tip end 2.

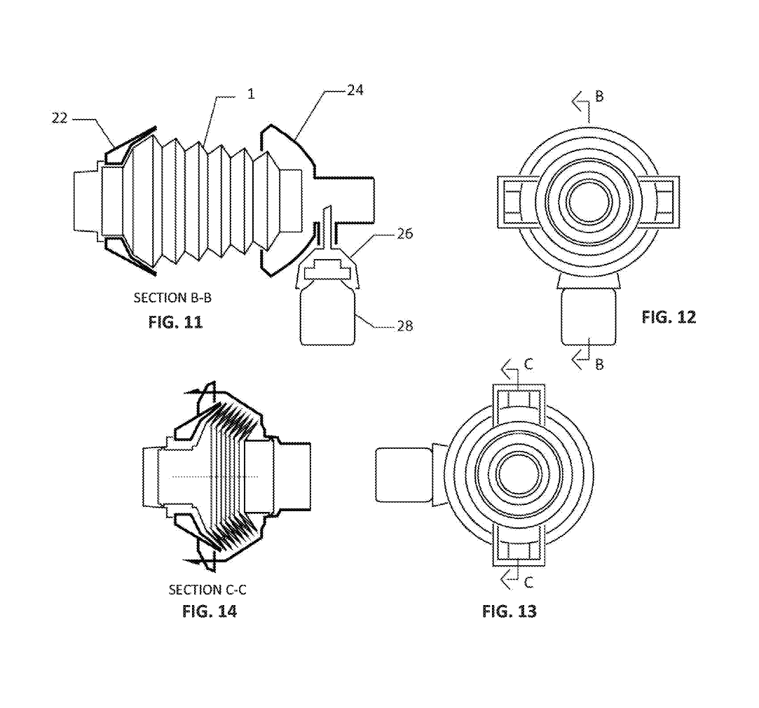

FIGS. 11-14 show the spacer chamber 10 of the present invention in use with a mechanical ventilation. A female connector 22 is connected to the tip end 2 of the body 1. The female connector 22 may include clamps to prevent the chamber to extend because of the pressure of the ventilator. A male connector 24 is connected to the base end 3 for air input into the chamber. The male connector 24 includes clamps for helping to hold the aerosol input device 26 that receives the aerosol device 28.

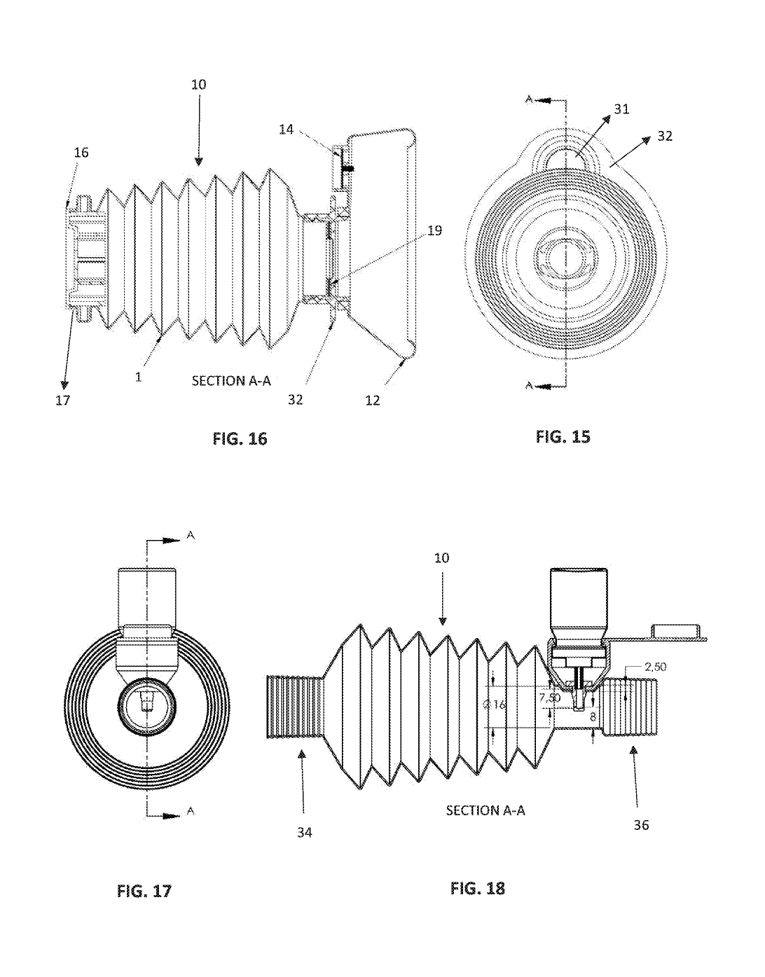

FIGS. 15-16 show the space chamber 10 including a flap 32 built into the base end 2. The flap 32 serves as a handle to help the patient to manipulate the extension of the body 1. The flap 32 is built into the base end 2 during the manufacturing process. Because the flap 32 is built into the base end, the flap 32 is made of the same material as the one piece body 1. The flap 32 may include a hole 31 adapted to accommodate a finger of the patient during the extension of the body 1.

FIGS. 17 and 18 show a spacer chamber according to an embodiment of the present invention showing the extended chamber in use with a mechanical ventilation and including a threaded base end 34 and a threaded tip end 36.

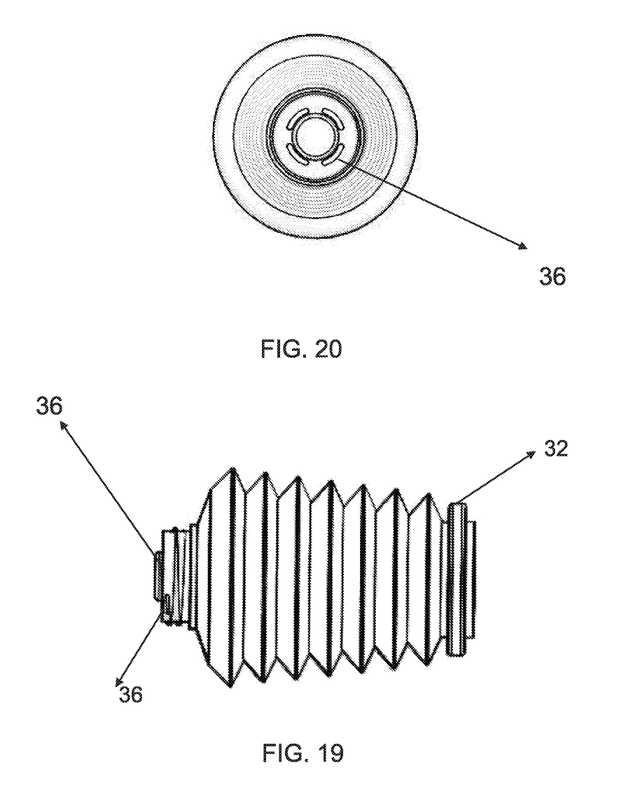

FIGS. 19-20 show the spacer chamber 10 including the flap 32, a threaded tip end, and orifices 36 in the threaded tip end 36. The orifices 36 allow the aerosol to pass through. In addition, the valve 14 covers the orifices 36 preventing blow inside the chamber.

What is claimed is:

1. A spacer chamber for administering a therapeutic agent spray comprising:
a one piece body 1 having a base end 3, a tip end 2, and a popoid section 4,
wherein the one piece body 1 is made entirely of a flexible material and has a shape of a cone;
wherein a diameter 3.1 of the base end 3 is larger than a diameter 2.1 of the tip end 2;
wherein the popoid section 4 includes a plurality of popoids 4.1, each popoid 4.1 includes a first set of frustoconical portions 4.2 facing opposite to a second set of frustoconical portions 4.3 to form a corrugation 4.4 in the body 1;
wherein the first and second set of frustoconical portions 4.2, 4.3 of the popoid 4.1 near to the base end 3 have a greater diameter than the diameter of the first and second set of frustoconical portions 4.2, 4.3 of the popoid 4.1 near to the tip end 2; and
wherein when the tip end 2 moves toward the base end 2, the frustoconical portions 4.3 maintain their shape, while the frustoconical portions 4.2 become inverted to fit within the associated frustoconical portions 4.3, thus, each popoid 4.1 collapses upon itself and each popoid 4.1 beginning from the tip end 2 collapses completely inside an adjacent popoid 4.1 shortening the length of the body 1.

2. The spacer chamber of claim 1, wherein the flexible material is polypropylene.

3. The spacer chamber of claim 1, wherein the base end and the tip end are threaded.

4. The spacer chamber of claim 1, wherein the base end includes a built in flap having a hole, the hole is adapted to receive a finger of a user to help in expanding the body.

5. The spacer chamber of claim 1, wherein the tip end includes a plurality of orifices.
